(19) United States
(12) Patent Application Publication
(10) Pub. No.: US 2005/0233330 A1
(43) Pub. Date: Oct. 20, 2005

(54) Method for identifying myelodysplastic syndrome-specific genes

(75) Inventor: Hiroyuki Mano, Utsunomiya-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co., Ltd., Osaka-shi (JP)

(21) Appl. No.: 10/824,536

(22) Filed: Apr. 15, 2004

(51) Int. Cl. C12Q 1/68

(52) U.S. Cl. 435/6

(57) ABSTRACT

Myelodysplastic syndrome (MDS) is a clonal disorder of hematopoietic stem cells (HSC). Clinical course of MDS can be divided into several stages; an indolent chronic phase termed “refractory anemia (RA)” or “RA with ringed sideroblasts”, and advanced stages including “RA with excess of blasts (RAEB)” and MDS-associated leukemia. Despite a relatively high incidence of MDS, there are few effective means to treat individuals at its advanced stages. DNA microarray would be a useful tool to clarify the molecular pathogenesis of, and to develop novel treatments against, MDS. However, a simple comparison with DNA microarray of bone marrow (BM) mononuclear cells from individuals at distinct stages of MDS would mainly lead to the identification of “pseudo-positive” genes whose expression alterations only reflect the difference in the proportion of MDS blasts within BM. To efficiently analyze the stage-progression mechanism in MDS, AC133 cell surface marker-positive HSC was purified from BM of healthy volunteers as well as 30 MDS patients, and used to compare the expression profiles of 2304 genes by microarray made by the inventor. The inventor succeeded in isolating sets of genes, expression of which was specific to either indolent stage or the advanced ones.

### FIELD OF THE INVENTION

The present invention relates to a method for identifying myelodysplastic syndrome (MDS)-specific genes. Furthermore, the present invention relates to methods of diagnosis for MDS using the genes, and diagnostic drugs used therefore. The present invention further relates to methods for identifying compounds for treating or preventing MDS and a drug comprising the identified compound as an active ingredient.

### BACKGROUND OF THE INVENTION

Myelodysplastic syndrome (MDS) is a clonal hematological disorder that mainly affects elderly people (Lowenthal, R. M. & Marsden, K. A. (1997) Int. J. Hematol. 65, 319-338). The characteristic feature to MDS is the presence of dysplasia in multiple lineages of blood cells, such as those of myeloid, erythroid and megakaryocyte/platelet lineages. It is therefore believed that MDS results from the malignant transformation at the level of pluripotent hematopoietic stem cells (HSC). Another important character to MDS is the co-existence of increased cellularity in bone marrow (BM) and cytopenia in peripheral blood (PB); a condition referred to as “ineffective hematopoiesis”. Immature BM cells of MDS patients may have a defect in the differentiation program or undergo apoptosis before giving rise to a sufficient number of progeny.

The clinical course of MDS is composed of several distinct phases (Harris, N. L., et al. (1999) J. Clin. Oncol. 17, 3835-3849). In the early, indolent stage, patients only suffer from cytopenia and may not require any specific treatment. Such patients are classified into “refractory anemia (RA)” or “RAwith ringed sideroblasts (RARS)”, depending on the absence or presence of ringed sideroblasts in BM. After experiencing the indolent phase for several (or even decades of) years, however, a part of the patients undergo transformation to a leukemic state. As the leukemic blasts increase in BM, patients are diagnosed to have “RA with excess of blasts (RAEB) (5-20% blasts in BM)”, and finally MDS-associated “acute myeloid leukemia (AML) (>20% blasts in BM)”. The malignant cells in such MDS-associated leukemia are refractory to chemotherapeutic agents, and the median survival duration of these patients is less than a year.

Therefore, to develop an effective treatment for MDS, it would be essential to clarify the molecular mechanism underlying the stage progression in MDS. Unfortunately, there exists only a limited amount of information on this issue. Point mutations in the RAS genes have been found in MDS cells. However, its significance as a prognostic value is still controversial (Horiike, S., et al. (1994) Leukemia 8, 1331-1336; Neubauer, A., et al. (1994) Leukemia 8, 638-641) Others have reported decrease in the expression of tumor-suppressor genes in MDS cells. Allelic loss of p53 gene and point mutations in its remaining allele has been reported in the advanced stages of MDS, albeit at a low frequency (Fenaux, P., et al. (1996) Semin. Hematol. 33, 127-138). Transcriptional suppression through promoter-silencing has been documented for another tumor-suppressor gene, p15^{INK4b} (Quesnel, B., et al. (1998) Blood 91, 2985-2990). However, it has not been proven whether these expression alterations are relevant to the stage progression in MDS.

DNA microarray enables monitoring the “transcriptome” of given cell types or tissues. Expression profile of thousands of genes can be simultaneously quantitated with this technology (Duggan, D. J., et al. (1999) Nat. Genet. 21, 10-14) Such large scale screening may allow to identify genes, whose expression changes in a stage-dependent manner during the clinical course of MDS. Extraction of these stage-specific genes would facilitate the molecular diagnosis of, as well as the prediction of stage progression for MDS, and would help to gain insights into the molecular events that drive the expansion of leukemic blasts in MDS.

However, the proportion of leukemic blasts varies widely among patients at different stages in MDS. Therefore, a simple comparison of BM mononuclear cells (MNC) among different patients using microarray is likely to generate a large number of pseudo-positive results. If, for instance, the transcriptome of BM MNC is compared between the individuals with RA and MDS-associated leukemia, one would mistakenly conclude according to the assay that any genes specific to immature blood cells are induced in MDS-associated leukemia. This “apparent” induction may only reflect the expansion of immature leukemic cell-population in the BM of MDS-associated leukemia, and the assay dose not correctly address whether mRNA copy number per cell is truly changed between the two individuals.

To exclude such “population-shift effect”, it would be effective to purify background-matched populations from both specimens prior to microarray analysis, and to compare the transcriptome between the purified fractions. Given the fact that malignant transformation takes place at HSC in MDS, the present inventor reasoned that HSC would be the good target for such BAckground-Matched Population (BAMP) screening (Miyazato, A., et al. (2001) Blood 98, 422-427) of MDS. Analyses with HSC should make it possible to directly compare the transcriptome of MDS blasts irrespective of the blast population size within BM or the differentiation ability of the blasts. For this purpose, the present inventor took advantage of an HSC-specific cell surface marker, AC133 (Hin, A. H., et al. (1997) Blood 90, 5002-5012), and started to purify and store AC133-positive HSC-like fractions from individuals with leukemia or leukemia-related disorders including MDS; a depository termed as “Blast Bank” (Miyazato, A., et al. (2001) Blood 98, 422-427). By using this Bank cells, the present inventor has already identified genes, expression of which is useful in the differential diagnosis between MDS-associated leukemia and de novo AML (Miyazato, A., et al. (2001) Blood 98, 422-427). Also clusters of genes whose expression is stage-dependent in chronic myeloid leukemia (CML) have been revealed (Ohmine, K., et al. (2001) Oncogene 20, 8249-8257).

### SUMMARY OF THE INVENTION

It is a goal of this invention to provide a method for identifying MDS-specific genes. As previously demonstrated by the present inventor, the BAMP screening with purified blasts has a less number of pseudo-positive data compared to those obtained from mononuclear cell (MNC) comparison (Miyazato, A., et al. (2001) Blood 98, 422-427; Ohmine, K., et al. (2001) Oncogene 20, 8249-8257). Thus, the present invention relates to a method for identifying MDS-specific genes utilizing the BAMP screening method, as well as methods of diagnosis for MDS using the genes, and drugs used therefore. Furthermore, the present invention relates to methods for identifying compounds for treating or preventing MDS.

One embodiment of the present invention relates to a method for identifying an MDS gene. More specifically, the present invention relates to a method for identifying an MDS gene comprising the steps of:
(a) detecting gene expressions in hematopoietic stem cells prepared from an MDS patient at a bad prognosis stage and a normal individual or patient at a good prognosis stage;
(b) comparing the gene expression in the hematopoietic stem cells between the MDS patient at a bad prognosis stage and the normal individual or patient at a good prognosis stage; and
(c) identifying a gene specifically overexpressed or underexpressed in the MDS patient at a bad prognosis stage. The hematopoietic stem cells wherein the gene expression is detected according to the present invention can be prepared from bone marrow aspirate. The preparation can be conducted using hematopoietic stem cell-specific cell surface marker as an index.

Another embodiment of the present invention provides a method of diagnosis for MDS. The present method of diagnosis comprises the steps of:
(a) detecting, in a tissue or cells prepared from a subject, expression of an MDS-specific gene; and
(b) comparing the expression detected in step (a) with that in a control tissue or control cells.

According to the method, the subject is judged to be at a risk of MDS if the expression detected in step (a) is significantly higher than the expression of the gene in the control tissue or control cells, where the gene is specifically expressed in an MDS patient. Furthermore, the subject is judged to be at a risk of MDS if the expression detected in step (a) is significantly lower than that in the control tissue or control cells, where the gene is specifically expressed in individuals free from MDS. The MDS gene whose expression is detected in the present method is not limited so long as it can be detected according to the method of identifying an MDS-specific gene of the present invention. For example, the expression of genes that were identified in Example, i.e., PIASy gene, LIM2 gene, NDUFV1 gene and PNMA2 gene, can be detected in the present method of diagnosis. To detect the expression of an MDS-specific gene in tissues or cells of the subject, for example, hematopoietic stem cells prepared from the subject can be used. The hematopoietic stem cells can be prepared from bone marrow aspirate of the subject using hematopoietic stem cell-specific cell surface marker as an index.

Furthermore, the present invention relates to a drug for diagnosing MDS. The present drug may comprise, as an active ingredient, an antibody binding to a protein encoded by an MDS-specific gene or a polynucleotide specifically hybridizing to a transcription product of an MDS-specific gene. Subjects can be diagnosed for MDS using such diagnostic drug through the detection of an expression product (transcription or translation product; mRNA, protein, etc.) of the MDS-specific gene in the tissue or cells of the subject. Herein, the MDS-specific gene whose expression is detected may be any gene so long as it can be identified by the method of identifying MDS-specific gene of the present invention, and includes PIASy gene, LIM2 gene, NDUFV1 gene, and PNMA2 gene.

Moreover, as another embodiment of the present invention a method of diagnosis for MDS which comprises the step of detecting, in a biological sample, a genetic polymorphism or mutation that causes abnormal expression of an MDS-specific gene or abnormal activity of a protein encoded by the gene. Herein, a subject from whom the biological sample was taken is judged to be at a risk of MDS if the genetic polymorphism or mutation is detected in the biological sample. The MDS-specific gene whose expression is detected may be any gene so long as it can be identified by the method of identifying MDS-specific gene of the present invention, and includes PIASy gene, LIM2 gene, NDUFV1 gene, and PNMA2 gene.

The present invention further relates to a method for identifying compounds for treatingand/orpreventingMDS. The phrase “compounds for treating and/or preventing MDS” refers to compounds that serve as drug candidates for treating or preventing MDS. Specifically, such compounds can be identified through the steps of:
(a) administering or contacting a test compound to a test animal or test cells; and
(b) detecting, in the test animal or test cells, expression of a MDS-specific gene. Herein, the test compound is judged to be a drug candidate compound for treating or preventing MDS if the test compound decreases the expression detected in step (b), where the gene is specifically expressed in an MDS patient. Furthermore, the test compound is judged to be a drug candidate compound for treating or preventing MDS if the test compound increases the expression detected in step (b), where the gene is specifically expressed in individuals free from MDS. Alternatively, compounds for treating or preventing MDS can be identified via the steps of:
(a) administering or contacting a test compound to a test animal or test cells harboring a reporter gene operably linked to the expression control region of an MDS-specific gene; and
(b) detecting, in the test animal or test cells, expression of the reporter gene. Herein, the test compound is judged to be a drug candidate compound for treating or preventing MDS if the test compound decreases the expression detected in step (b), where the MDS-specific gene is specifically expressed in an MDS patient. Furthermore, the test compound is judged to be a drug candidate compound for treating or preventing MDS if the test compound increases the expression detected in step (b), where the gene is specifically expressed in individuals free from MDS. Furthermore, as an alternate method for identifying compounds for treating or preventing MDS, a method comprising the steps of:
(a) contacting a test compound with a protein encoded by an MDS-specific gene; and
(b) detecting activity of the protein. According to this method, the test compound is judged to be a drug candidate compound for treating or preventing MDS if the test compound decreases the activity detected in step (b), where the gene is specifically expressed in an MDS patient. Furthermore, the test compound is judged to be a drug candidate compound for treating or preventing MDS if the test compound increases the activity detected in step (b), where the gene is specifically expressed in individuals free from MDS. The MDS-specific gene used in these three methods for identifying drug candidate compounds is not limited and may be any gene that can be identified through the method for identifying an MDS-specific gene of the present invention. For example, PIASy gene, LIM2 gene, NDUFV1 gene and PNMA2 gene are included as the gene.

Furthermore, the present invention relates to a drug for treating or preventing MDS that comprises, as an active ingredient, a compound identified by any of the above-mentioned methods for identifying a drug candidate compound.

According to the knowledge of the applicant, this is the first report for large-scale expression profiling of fresh MDS samples, especially with fractionated MDS blasts. In conclusion, the microarray analysis with the purified Blast Bank samples was demonstrated to be a highly useful system to identify molecular markers for the various stages of MDS as well as to provide insight into the molecular mechanism of transformation. Through the present method, MDS-specific genes can be efficiently identified. The MDS-specific genes identified through the present method can be used for diagnosing MDS using mutation or expression of the MDS-specific gene as an index. MSD-specific genes specific to each of the MDS stages are identified through the method of identifying MDS-specific gene of the present invention. Therefore, diagnosis using these genes enables prediction of stage progression of MDS. Furthermore, the genes identified according to the present method can be used for selecting drug candidate compounds for treating or preventing MDS.

### BRIEF DESCRIPTION OF THE DRAWINGS

FIG. 1(A) Hierarchical clustering of 2304 genes based on their expression profiles in Blast Bank samples derived from a pooled healthy control sample (CTRL) and 30 individuals with MDS consisting of 11 RA, 5 RAEB and 14 MDS-associated leukemia (Leukemia) patients. Each column represents a single gene on the microarray, and each row a separate patient sample. The fluorescence intensity of respective gene was normalized relative to the median fluorescence value of all spots in each hybridization, and the normalized value is shown color-coded as indicated at the left. The positions of a cluster of RA-specific genes (cluster #1) and that of MDS-leukemia-specific genes (cluster #2) are indicated. (B) Mean expression value for each gene was calculated for RA, RAEB and MDS-associated leukemia stages, and used to generate a dendrogram, “average tree”. The presence of clusters of genes whose expression is specific to subgroups has been revealed. (C) Comparison of gene expression between the good prognosis (healthy control and RA) and bad prognosis (RAEB and MDS-associated leukemia) groups. The mean expression value for each gene was calculated within the good prognosis or the bad prognosis group, and was used to demonstrate the alterations of gene expression level between the groups. Each line corresponds to a single gene and is colored according to the mean expression level of the gene in the good prognosis group. The hypothesized “bad prognosis-specific gene” is shown in blue.

FIG. 2(A) Identification of prognosis-dependent genes. Expression profiles of 11 bad prognosis-specific genes (upper panel) and 7 good prognosis-specific genes (lower panel) are shown color-coded as indicated at the bottom. Each row corresponds to a single gene, with the columns indicating the corresponding expression level in different AC133+cells obtained from a healthy control (CTRL), RA, RAEB and MDS-associated leukemia (Leukemia) samples. The similarity of expression profiles among the bad prognosis-specific or the good prognosis-specific genes is indicated by the purple-colored or the blue-colored dendrogram, respectively, at the left. The names and accession numbers of the genes as well as expression intensity data for the genes are available as a Supplementary Information through the website of PNAS. (B) Quantitation of PIASY transcript in MDS blasts. Complementary DNAs were prepared from the blasts of 37 individuals consisting of 2 healthy volunteers (CTRL), 13 RA, 9 RAEB and 13 MDS-associated leukemia (Leukemia) patients, and were subjected to real-time PCR with primers specific for PIASY or β-actin genes. The ratio of the abundance of the PIASY transcript to that of β-actin mRNA was calculated as 2ⁿ, where n is the C_{T} value for β-actin cDNA minus the C_{T} value of the PIASy cDNA.

FIG. 3(A) Conditional expression of PIASy. 32D cells were infected with MX-tetOFF (Mock) or MX-tetOFF/PIASy-F (PIASy) retrovirus, and cultured under the presence of IL-3, blasticidin-S and tetracycline. The blasticidin-S-resistant mass culture was left in the same condition (−), or incubated in the medium containing β-estradiol in place of tetracycline (+). After an overnight culture, cells were harvested and subjected to the immunoblot analysis with the antibodies to FLAG tag. As indicated at the right, expression of PIASy-F was only detectable in the MX-tetOFF-infected cells under the presence of β-estradiol. (B) 32D cells infected with MX-tetOFF (circle) or MX-tetOFF/PIASy-F (triangle) were cultured in RPMI1640/FBS supplemented with G-CSF, either under the non-induced (open symbol) or induced (closed symbol) condition. Total cell number (left panel) and the viability as judged by the trypan-blue dye exclusion method (right panel) of each fraction was counted at every other day, and shown as a graph.

FIG. 4(A) 32D cells infected with MX-tetOFF (Mock) or MX-tetOFF/PIASy-F (PIASy) were incubated for 8 days under the induced condition, either with IL-3 or G-CSF. Cytospin preparation of each fraction was stained with the Wright-Giemsa solutions. Original magnification ×200. (B) 32D cells infected with MX-tetOFF (Mock) or MX-tetOFF/PIASy-F (PIASy) were incubated for 8 days with G-CSF under the non-induced [tet (+), β-est (−)] or induced [tet (−), β-est (+)] condition. Cells were then stained with Annexin V-FITC and PI, and subjected to flow cytometry. The percentages of apoptotic cells (Annexin V-positive and PI-negative) are designated. (C) 32D cells infected with MX-tetOFF (Mock) or MX-tetOFF/PIASy-F (PIASy) were cultured under the induced condition, either with IL-3 or G-CSF. The proportion of differentiated cells was then evaluated by flow cytometry with the antibodies to a granulocyte-specific marker, Gr-1. The percentage of Gr-1⁺ cells in each fraction is shown.

### DETAILED DESCRIPTION OF THE INVENTION

Any publications referenced herein are hereby incorporated by reference in this application in order to more fully describe the state of the art to which the present invention pertains.

In this application, the inventor has compared the expression profiles of 2,304 genes, including those coding for cell surface proteins, signaling components and transcriptional factors, among the Blast Bank samples obtained from 30 MDS patients (11 with RA, 5 with RAEB and 14 with MDS-associated leukemia) and healthy volunteers. Interestingly, a set of genes, expression of which was high in control or RA samples but declined in the advanced stages, extracted by BAMP screening included that for PIASy, a potential inhibitor for STAT1 (Liu, B., et al. (2001) Proc. Natl. Acad. Sci. USA 98, 3203-3207). Further investigation has revealed that expression of PIASy induces apoptosis in mouse myeloid cell line, 32D. Decrease of PIASy expression may, therefore, play a role in the outgrowth of leukemic blasts, leading to the stage progression in MDS. These results indicate that PIASy functions as a “tumor-suppressor” and that it prevents stage progression in MDS. This shows that the identification of genes utilizing BAMP screening is adapted to identify MDS-specific genes.

Thus, the present invention relates to a method for identifying MDS-specific genes utilizing the BAMP screening method. More specifically, the present method comprises the steps of: (a) detecting gene expressions in HSCs prepared from an MDS patient at a bad prognosis stage and a normal individual or MDS patient at a good prognosis stage; (b) comparing the gene expression in the HSCs between the MDS patient at a bad prognosis stage and the normal individual or MDS patient at a good prognosis stage; and (c) identifying a gene specifically expressed (overexpressed) or suppressed (underexpressed) in the MDS patient at a bad prognosis stage.

Herein, the HSCs are preferably prepared from bone marrow (BM) MNC collected from BM aspirates using hematopoietic stem cell-specific cell surface marker as an index. The “HSC-specific cell surface marker” is not restricted so long as it is a surface protein specific to the HSC, and includes, in addition to AC133, KDR (GenBank accession number: NM_{—}002253) and KIT (NM_{—}000222). HSCs can be, for example, collected from BM MNC using antibodies against an HSC-specific cell surface marker. The phrase “MDS patient at a bad prognosis stage” refers to patients diagnosed to have “Refractory anemia with excess of blasts (RAEB)” or MDS-associated “acute myeloid leukemia (AML)”. Patients are judged to be at the stage of RAEB and MDS-associated AML when the blasts in BM are 5-20% and more than 20%, respectively. On the other hand, the phrase “normal individual or an MDS patient at a good prognosis stage” refers to those who are healthy or who are at the stage of “Refractory anemia (RA)”. According to the present method, human is preferred as the patient or subject whose gene expression is detected, but the present is not restricted thereto and any mammalian animal may be used as the target. According to the present invention, expression of the genes in the HSC are preferably detected using an oligonucleotide microarray that contains oligonucleotides based on genes that encode transcription factors, membrane proteins, proteins involved in cell signaling and redox regulation, and so on. First, total RNA is extracted from HSC, for example, by the acid guanidium method, and amplified to convert them to double-stranded cDNAs. cDNAs can be prepared from poly (A)+ RNAs by the oligocap method (Maruyama M. and Sugano S., Gene 138: 171-4 (1994)), or utilizing cDNA synthesis system (GIBCO BRL). Then, the cDNAs are converted to labeled-cRNAs for the hybridization with a microarray. The cRNAs may be labeled with any markers including enzymes (e.g., peroxidases such as horseradish peroxidase, alkaline phosphatase, β-D-galactosidase, glucose oxidase, glucose-6-phosphate dehydrogenase, alcohol dehydrogenase, malate dehydrogenase, penicillinase, catalase, apoglucose oxidase, urease, luciferase, acetylcholine esterase, etc.); fluorescent substances (e.g., fluorescein isothiocyanate, ficobiriprotein, rare earth metal chelates, dancylchloride, tetramethylrhodamine isothiocyanate, etc.); chemical luminescence substances; biotin; avidin; isotopes (e.g., ³H, ¹⁴C, ¹²⁵I, ¹³¹I, etc.); and such. Isotopes and fluorescent substances can be detected alone, whereas enzymes, chemical luminescence substances, biotin and avidin don't emit any detectable signal without the aid of other substances. Thus, for example, enzyme labels can be detected by reacting them with detectable substrates. The activity of an enzyme label can be measured by colorimetry, fluorescent method, bioluminescent method, chemical luminescent method, and such. When biotin is used as the label, the measurement can be conducted with the aid of avidin, streptavidin, etc.

Such genes isolated according to the present method can be used for the diagnosis of MDS. Specifically, the present invention relates to a method of diagnosis for MDS, said method comprising the steps of: (a) detecting, in tissue or cells prepare from a subject, expression of an MDS-specific gene; and (b) comparing the expression detected in step (a) with that in a control tissue or control cells, wherein the subject is judged to be at a risk of MDS if the expression detected in step (a) is significantly higher than the expression of the gene in the control tissue or control cells, where the gene is specifically expressed in an MDS patient, or if the expression detected in step (a) is significantly lower than that in the control tissue or control cells, where the gene is specifically expressed in individuals free from MDS.

According to the method, it is preferred to use HSCs prepared from BM aspirate using HSC-specific cell surface markers as described above. Furthermore, the MDS-specific gene may be any gene that can be identified according to the method for identifying an MDS-specific gene of the present invention, and includes PIASy gene, LIM-Hox2 (LH2) gene, NADH-ubiquinone oxidoreductase flavoprotein 1 (NDUFV1) gene and paraneoplastic antigen MA2 (PNMA2) gene. According to the present diagnostic method, the expression of multiple MDS-specific genes may be detected. The detection of multiple MDS-specific genes is expected to enhance the reliability of the diagnosis. Moreover, in addition to the expression of the MDS-specific gene, the expression of genes serving as control may be detected. The genes used as control is not limited in any way. However, it is preferred to use genes that are expressed MDS independently and universally as the control. For example, a gene encoding β-actin may be used as the control.

PIASy was identified as a binding protein to, and a suppressor for the activity of, STAT1 (Liu, B., et al. (2001) Proc. Natl. Acad. Sci. USA 98, 3203-3207). In addition to such a regulatory role for STAT proteins, recent reports indicate a wide spectrum of PIAS functions than previously expected, such as modification of the activity of androgen receptor (Gross, M., et al. (2001) Oncogene 20, 3880-3887) or p53 (Nelson, V., et al. (2001) Apoptosis 6, 221-234), or sumoylation of a transcriptional factor, LEF1 (Sachdev, S., et al. (2001) Genes Dev. 15, 3088-3103). Interestingly, Liu et al. have shown that the forced expression of PIASy in human kidney 293T cells is accompanied with induction of apoptosis (Liu, B. & Shuai, K. (2001) J. Biol. Chem. 276, 36624-36631), suggesting the proapoptotic activity of PIASy. The encoded amino acid sequence and the nucleotide sequence of PIASy are registered with the Accession Nos. NPO₅₇₂₃₃ and BQ263140 on the database, respectively.

LH2 is a homeobox-containing transcriptional factor. Although neither target genes nor the in vivo functions for LH2 have been identified, possible involvement of LH2 in mitogenic signaling has been proposed by the facts that the LH2 gene is aberrantly expressed in chronic myeloid leukemia (Wu, H. K., et al. (1996) Oncogene 12, 1205-1212) and that LH2 is expressed in immature, but not in mature B-cells in vivo (Xu, Y., et al. (1993) Proc. Natl. Acad. Sci. USA 90, 227-231). The encoded amino acid sequence and the nucleotide sequence of LH2 are registered with the Accession Nos. P50458 and AI885630 on the database, respectively.

NDUFV1 is a component of NADH: ubiquinone oxidoreductase that is involved in mitochondrial electron transport (Ali, S. T., et al. (1993) Genomics 18, 435-439). Increased expression of NDUFV1 may therefore reflect the increased mitochondrial respiratory rate within the transformed blast cells. The encoded amino acid sequence and the nucleotide sequence of NDUFV1 are registered with the Accession Nos. AF053069 and NM_{—}007103 on the database, respectively (Biochem. Biophys. Res. Commun. 245 (2): 599-606 (1998)).

Paraneoplastic antigen MA2 (PNMA2) was originally identified as a serologic marker produced from testicular cancer cells (Voltz, R., et al. (1999) N. Engl. J. Med. 340, 1788-1795), and may play as an antigen causing paraneoplastic syndromes. The encoded amino acid sequence and the nucleotide sequence of PNMA2 are registered with the Accession Nos. NP_{—}009188 and NM_{—}007257 on the database, respectively.

The expression of an MDS-specific gene can be detected by Western blotting, Immunoprecipitation, ELISA and such, using an antibody that binds to a protein encoded by an MDS-specific gene identified by the method for identifying an MDS-specific gene of the present invention and by extracting proteins from tissue and cells. Herein, the term “antibody” refers to any antibody so long as it specifically detects proteins encoded by an MDS-specific gene, and includes monoclonal antibodies including chimeric antibodies; polyclonal antibodies; multispecific antibodies, such as bispecific antibodies and diabodies (EP404097; WO93/11161; Hollinger et al., Proc. Natl. Acad. Sci. USA 90: 6444-8(1993)); and antibody fragments retaining the antigen binding activity including Fab, Fab′, F(ab′)₂ and Fv (e.g., sFV (Pluckthun, The Pharmacology of Monoclonal Antibodies, vol. 113., Rosenburg and Moore eds. Springer-Verlag, New York, p269-315 (1994)).

Polyclonal antibodies may be prepared by immunizing nonhuman mammals, such as rabbits and mice, subcutaneously or interperitoneally with the protein encoded by the MDS-specific gene of the present invention or fragments thereof (Current protocols in Molecular Biology edit. Ausubel et al. (1987) Publish. John Wiley & Sons., Section 11.12-11.13) The protein used for immunization may be bound to keyhole limpet hemocyanin, serum albumin, bovine thyroglobulin or soybean trypsin inhibitor through bifunctional agents and derivatizing agents including maleimidebenzoyl sulfosuccinimide ester (conjugation through cystein residue) N-hydrosuccinimide (conjugation through lysine residue), glutaraldehyde, succinic anhydide, thionyl chloride, and so on according to needs.

On the other hand, monoclonal antibodies can be produced by preparing hybridoma cell via fusion of spleen cells, which is derived from animals immunized with a protein encoded by the MDS-specific gene of the present invention or fragments thereof, and myeloma cells (Current protocols in Molecular Biology edit. Ausubel et al. (1987) Publish. John Wiley & Sons., Section 11.4-11.11). Alternatively, monoclonal antibodies may be prepared in vitro by the phage display method (Hawkins et al., J. Mol. Biol. 254: 889-96 (1992); Lowman et al., Biochemistry 30(45): 10832-8 (1991)). Furthermore, methods for altering antibodies are well known in the art (see, for example, U.S. Pat. No. 5,994,511), and modified antibodies may also be used for the detection of MDS-specific gene expression.

Alternatively, the expression of an MDS-specific gene can be detected using a polynucleotide specifically hybridizing to a transcription product (e.g., mRNA) of an MDS-specific gene identified by the method for identifying an MDS-specific gene of the present invention. There is no restriction on the type of the polynucleotides used in this method so long as they specifically hybridize to the MDS-specific gene identified according to the present method, and include genes such as PIASy, LH2, NDUFV1, and PNMA2, and fragments thereof. Preferably, the polynucleotides have a chain length of at least 15 bp, more preferably longer than 100 bp, furthermore preferably longer than 500 bp. Generally, such polynucleotides have a chain length within 3000 bp, and more preferably within 2000 bp. Moreover, antisense nucleic acids, ribozymes and siRNA that digest mRNAs of the MDS-specific genes are included as such polynucleotides. Polynucleotides such as antisense nucleic acid, ribozyme and siRNA can be also used for suppressing the expression of the MDS-specific gene. Such polynucleotides that suppress gene expression can be prepared according to conventional methods.

Such polynucleotides used for the detection of the present invention can be prepared according to the method for identifying MDS-specific genes of the present invention. Further, by sequencing the identified genes according to conventional methods, the polynucleotides can be prepared from sources (e.g., HSC) containing the MDS-specific genes with probes and/or primers by hybridization (Current Protocols in Molecular Biology edit. Ausubel et al. (1987) Publish. John Wiley & Sons Section 6.3-6.4) or PCR to further amplify the gene. Alternatively, the genes and fragments thereof may be constructed chemically.

The polynucleotides used in the present invention don't have to be completely complementary to the MDS-specific genes of the present invention, so long as they specifically hybridize to the genes. Such polynucleotides may be obtained by site-specific mutagenesis (Current Protocols in Molecular Biology edit. Ausubel et al. (1987) Publish. John Wiley & Sons Section 8.1-8.5), PCR (Current Protocols in Molecular Biology edit. Ausubel et al. (1987) Publish. John Wiley & Sons Section 6.1-6.4), normal hybridization method (Current Protocols in Molecular Biology edit. Ausubel et al. (1987) Publish. John Wiley & Sons Section 6.3-6.4), and so on.

A stringent condition for hybridization includes normally a wash condition “1×SSC, 37° C.”. A more stringent condition would be a wash condition “0.5×SSC, 0.1% SDS, 42° C.”, and a much more stringent condition would be “0.1×SSC, 0.1% SDS, 65° C.”. The stringent the condition, the higher the homology of the obtained polynucleotide to the probe sequence. However, the hybridization conditions mentioned above are just examples, and it should be understood that those skilled in the art can select an appropriate condition for hybridization taking the nucleotide sequence, concentration, and length of the probe; reaction time; reaction temperature; concentration of the reagent; and such into consideration. The detection using polynucleotides of the present invention can be conducted following the procedure for hybridization (Current Protocols in Molecular Biology edit. Ausubel et al. (1987) Publish. John Wiley & Sons Section 6.3-6.4) or PCR including PCR-SSCP and such.

Thus, the present invention provides drugs for diagnosing MDS, said drug comprising, as an active ingredient, a molecule selected from the group consisting of: (a) an antibody binding to a protein encoded by an MDS-specific gene identified by the method for identifying an MDS-specific gene described above; and (b) a polynucleotide specifically hybridizing to a transcription product of an MDS-specific gene identified by the method for identifying an MDS-specific gene described above. The diagnostic drug of the present invention may comprise one of the above-described antibody or polynucleotide, or may include multiple antibodies or polynucleotides described above. Alternatively, it may comprise a combination of the above-described antibodies and polynucleotides. A much reliable diagnostic result can be expected by detecting the expression of multiple MDS-specific genes. Such drugs may be used in the method of diagnosis for MDS described above.

Furthermore, a method of diagnosis for MDS comprising the step of: (a) detecting, in a biological sample, a genetic polymorphism or mutation that causes abnormal expression of an MDS-specific gene identified by the method for identifying an MDS-specific gene described above or abnormal activity of a protein encoded by the gene; wherein a subject from whom the biological sample was taken is judged to be at a risk of MDS if such genetic polymorphism or mutation is detected.

A polymorphism includes mini-satellite DNA, micro-satellite DNA, single nucleotide polymorphism (SNP), and so on. Such polymorphisms of an MDS-specific gene can be determined by sequencing the MDS-specific gene regions and regions related to its expression (e.g., promoter region, operator region) of healthy individuals and patients affected with MDS; and then comparing the determined sequences to detect polymorphic sites. Then, the allele frequency of the detected polymorphic sites is calculated. Those polymorphic sites which existence is significantly increased in patients can be determined to be related to MDS.

Abnormal expression of an MDS-specific gene may be caused, for example, by insertion of a stop codon, mutation of amino acid residues within the functional domain, and mutation of amino acid residues that cause conformational changes of the protein encoded by the gene. Thus, the biological sample used for the present invention is suitably a DNA sample comprising genomic DNA of the subject. Such DNA samples are exemplified by blood, urine, sperm, medulla fluid, visceral fluid, tissue such as liver, hair, etc. For example, a DNA sample is prepared from peripheral blood leukocyte using QIAapDNA blood kit (QIAGEN). Then, PCR is performed using the prepared DNA sample as the template and primers that are designed to amplify the target sites. Finally, the nucleotide sequence of the obtained PCR product is determined. It is preferred to use one of the primers used in the above PCR as a sequencing primer to determine the nucleotide sequence. The target site to be amplified according to the present method may be a polymorphic site of an MDS-specific gene, the whole MDS-specific gene or a part of the MDS-specific gene.

Alternatively, PCR-SSCP (single-strand conformation polymorphism; Genomics 12(1): 139-46 (1992); Oncogene 6(8): 1313-8 (1991); PCR Methods Appl. 4(5): 275-82 (1995)) can be conducted to detect a genetic polymorphism or mutation that causes abnormal expression of an MDS-specific gene. According to PCR-SSCP, the presence of a mutation in a DNA fragment due to even a single point mutation, deletion, insertion, and such can be detected by detecting the changes in the mobility of the target single-stranded DNA on a polyacrylamide gel by electrophoresis. The method is based on the fact that a single-stranded DNA dissociated from a double-stranded DNA forms a unique higher conformation, depending on respective nucleotide sequence. Complementary single-stranded DNAs having the same chain length of the dissociated DNA strand shift to different positions due to the difference of the respective higher conformations after electrophoresis on a polyacrylamide gel without a denaturant. Furthermore, the detection can be conducted based on methods like Restriction Fragment Length Polymorphism (RFLP) method and PCR-RFLP method that utilize the difference of the length of the restriction fragment of a genomic DNA due to the difference in the nucleotide sequence.

Alternatively, denaturing gradient gel electrophoresis (DGGE) can be used to detect polymorphism of a gene. First, the target site of the MDS-specific gene is amplified by PCR, electrophoresed on a polyacrylamide gel with gradient concentration of denaturant, such as urea; and the result is compared with that of a healthy individual. A polymorphism can be identified by detecting the difference in mobility of the DNA fragments, since the mobility speed of a fragment with mutations slows down to an extreme degree due to the separation into single-stranded DNAs at the part of the gel where the concentration of the denaturant is lower.

In addition, the detection can be conducted following the method of Allele Specific Oligonucleotide (ASO) hybridization. According to this method, first an oligonucleotide comprising a nucleotide sequence, wherein a polymorphism is predicted to exist, is prepared, and then is hybridized with a DNA sample. The efficiency of hybridization is reduced due to the existence of a polymorphic nucleotide that is different from the oligonucleotide in the sample DNA used for hybridization. The decrease of the hybridization efficiency can be detected by the Southern blotting method; methods which utilize specific fluorescent reagents with a characteristic to quench by intercalation into the gap of the hybrid; and such.

Furthermore, the detection may be also achieved by the ribonuclease A mismatch truncation method. Specifically, a region containing a target site of the MDS-specific gene is amplified by PCR and the like, and the amplified fragments are hybridized with labeled RNAs. Herein, the RNAs are prepared from cDNAs of a healthy individual (healthy-type cDNA) After cleaving with ribonuclease A sites of the hybrid that form a single-stranded conformation due to the existence of a nucleotide which is different from healthy type, a polymorphism can be detected by autoradiography and such.

Another embodiment of the method of diagnosis for MDS of the present invention comprises the step of detecting abnormal activity of a protein encoded by the MDS-specific gene. If the abnormal activity is caused by a different conformation of the protein, then such abnormal activity can be measured by methods including SDS polyacrylamide electrophoresis method; methods utilizing antibodies binding to the protein such as Western blotting method, dot-blotting method, immunoprecipitation method, enzyme-linked immunosorbent assay (ELISA) and immunofluorescence. Alternatively, the abnormal activity of a protein encoded by the MDS-specific gene can be detected through direct detection of the activity of the protein. For example, PIASy, one of the MDS-specific genes identified in the present invention is known to bind to and suppress the activity of STAT1. Thus, abnormal activity of PIASy may be detected by examining the binding ability of PIASy to STAT1 or STAT1 activity suppressing ability of PIASy. In addition, PIASy is known to have activities like modifying the activity of androgen receptor and p53, sumoylating LEF1, and inducing apoptosis in 293T cells. Thus, such activities may be used to detect abnormal activity of PIASy.

Furthermore, the present invention relates to a method for identifying compounds for treating or preventing MDS. Compounds that serve as candidates for agents for treating or preventing MDS can be identified by, for example: (a) administering or contacting a test compound to a test animal or test cells; and (b) detecting, in the test animal or test cells, expression of an MDS-specific gene identified by the method for identifying an MDS-specific gene of the present invention. The test compound is judged to be a drug candidate compound for treating or preventing MDS if the test compound decreases the expression detected in step (b), where the gene is specifically expressed in an MDS patient. Furthermore, the test compound is judged to be a drug candidate compound if the test compound increases the expression detected in step (b), where the gene is specifically expressed in individuals free from MDS.

Herein, a test compound can be any compound including culture supernatant of microorganisms; extracts of cells and tissues; gene library expression products; synthesized low molecular weight compounds; synthesized peptides; natural compounds derived from plants and marine organisms; random peptide groups produced by the phage-display method (J. Mol. Biol. 222: 301-10 (1991)); etc. Such compounds include the above-described antibodies against proteins encoded by an MDS-specific gene, and polynucleotides, such as antisense, ribozyme and siRNA, against the gene. A test animal or test cell may be any kind of animal or cell so long as it expresses an MDS-specific gene, which is specifically expressed in an MDS patient, or shows a suppressed expression of an MDS-specific gene, which is specifically expressed in individuals free from MDS. The administration of the compound to the animal may be conducted orally or parenterally following methods well established in the art. When contacting the test compound with the test cells, the cells may be immobilized on a carrier.

The step (b) of the present identification method can be performed according to the same procedure for detecting an MDS-specific gene of the method of diagnosis for MDS.

Alternatively, the method for identifying compounds for treating or preventing MDS can be conducted by: (a) administering a test compound to a test animal or test cells harboring a reporter gene operably linked to the expression control region of an MDS-specific gene identified by the method for identifying an MDS-specific gene of the present invention or contacting the test compound with the test animal or test cells; and (b) detecting, in the test animal or test cells, expression of the reporter gene. The test compound is judged to be a drug candidate compound for treating or preventing MDS if the test compound decreases the expression detected in step (b), where the MDS-specific gene is specifically expressed in an MDS patient. Furthermore, the test compound is judged to be a drug candidate compound if the test compound increases the expression detected in step (b), where the gene is specifically expressed in individuals free from MDS.

Herein, a reporter gene may include genes encoding, for example, luciferase, catalase, β-galactosidase, green fluorescent protein (GFP), and so on. The expression control region of an MDS-specific gene can be obtained from a genomic DNA library using the gene as a probe. Such an expression control region usually comprises a promoter sequence, ribosome-binding site, and so on. The test animal or test cells used in the method is not restricted so long as it recognizes the expression control region of the MDS-specific gene. However, mammal cells, such as COS cell, CHO cell and HEK 293 cell, can be mentioned as preferable examples.

To perform the method, first, a vector comprising a reporter gene operably linked to the expression control region of an MDS-specific gene is constructed. Then, the test animal or test cells are transformed with the vector, and any preferable test compound including those mentioned above are either administered or contacted to the animal or cells. Next, the expression of the reporter gene is measured according to conventional methods.

Furthermore, the method for identifying compounds for treating or preventing MDS can be also conducted by: (a) contacting a test compound with a protein encoded by an MDS-specific gene identified by the method for identifying an MDS-specific gene described above; and (b) detecting activity of the protein. The test compound is judged to be a drug candidate compound for treating or preventing MDS if the test compound decreases the activity detected in step (b), where the gene is specifically expressed in an MDS patient. Furthermore, the test compound is judged to be a drug candidate compound if the test compound increases the activity detected in step (b), where the gene is specifically expressed in individuals free from MDS.

Moreover, the present invention relates to drugs for treating or preventing MDS, said drug comprising, as an active ingredient, a compound identified by the method for identifying compounds for treating or preventing MDS described above.

Such drugs for treating or preventing MDS can be administered as a pharmaceutical composition prepared according to conventional pharmaceutical methods, in addition to directly administering the compound itself to a patient. For example, it can be formulated into a form suitable for oral or parenteral administration, such as tablet, pill, powder, granule, capsule, troche, syrup, liquid, emulsion, suspension, injection including liquid injection and suspension injection, suppository, inhalant, percutaneous absorbent, eye drop, eye ointment, obtained by mixing the active ingredient with a pharmacologically acceptable support. Pharacologically acceptable support can be exemplified by compounds such as excipient, binder, disintegrator, flavor, corrigent, emulsifier, diluent and solubilizer.

Administration to a patient can be typically carried out bymethods known in the art including intra-arterial injection, intravenous injection, subcutaneous injection, and such. Although the dosage varies depending on the weight, age, sex and symptom of the patient, administration method, and the like, one skilled in the art can appropriately select the appropriate dose. Furthermore, if the compound can be encoded by DNA, gene therapy can be also carried out.

Gene therapy can be carried out, for example, by inserting the DNA into a vector, which include, for example, viral vectors such as retroviral vectors, adenoviral vectors, adeno-associated viral vectors; and non-viral vectors such as liposomes; and soon. The objective DNA can be administered to a patient by ex vivo methods and in vivo methods utilizing such vectors.

Having generally described this invention, as disclosed and claimed herein, the following specific exemplary support is provided to demonstrate the functionality of the disclosed and claimed methods. However, this invention should not be interpreted as being limited to the specifics of the following Examples. Rather, the scope of this invention should be determined through consultation of the claims appended hereto and equivalents thereof.

### EXAMPLE 1

### Transcriptome of MDS Samples

Expression profiles of 2304 human genes were obtained for the Blast Bank samples purified from 11 patients with reflactory anemia (RA), 5 patients with RA with excess of blasts (RAEB) and 14 patients with myelodysplastic syndrome (MDS)-associated leukemia. First, bone marrow (BM) aspirates were obtained from subjects with written informed consent, and used to prepare mononuclear cells (MNC). Cells were then labeled with anti-AC133 (hematopoietic stem cell (HSC)-specific cell surface marker) MicroBeads (Miltenyi Biotec, Auburn, Calif.), and loaded onto a miniMACS magnetic cell separation column (Miltenyi Biotec) according to the manufacturer's protocol. The resulting AC133⁺ cell fractions were divided into portions and stored at −80° C. as the Blast Bank samples. AC133⁺ cells were also purified from BM MNC of two healthy volunteers and mixed for use as a “healthy control” sample. The purity of each sample was confirmed by Wright-Giemsa staining. When a sufficient number of AC133⁺ cells was obtained, the purity was also evaluated by flow cytometric analysis with antibodies to AC133, CD34 and CD38 (BD Biosciences, San Jose, Calif.). A total of 31 samples were thus subjected to the microarray experiments as described below.

Total RNA was extracted from Blast Bank samples by the acid guanidinium method. RNA was then subjected to two rounds of amplification (Van Gelder, et al. (1990) Proc. Natl. Acad. Sci. USA 87, 1663-1667), and the fidelity of the RNA amplification procedure was confirmed as previously described (Ohmine, K., et al. (2001) Oncogene 20, 8249-8257). One microgram of the amplified cRNA was then converted to double-stranded cDNA, followed by synthesis of biotin-labeled cRNA with the use of the ExpressChip labeling system (Mergen, San Leandro, Calif.). The labeled cRNA was allowed to hybridize with an oligonucleotide microarray (HO-3) that contains oligonucleotides based on genes that mostly encode transcription factors as well as with the array made by the inventor containing oligonucleotides for membrane proteins, and proteins involved in cell signaling or redox regulation (both obtained from Mergen). A total of 2,304 genes were studied for the expression profiling.

The microarrays were then incubated consecutively with streptavidin, anti-streptavidin first antibody, and finally Cy3-conjugated second antibody (all from Mergen) according to the manufacturer's instruction. Detection of the signals and analysis of the digitized data was carried out with a 418 array scanner (Affymetrix, Santa Clara, Calif.) and GeneSpring 4.1.0 software (Silicon Genetics, Redwood, Calif.), respectively. In the hierarchical clustering analysis, similarity was measured by the standard correlation with a separation ratio of 0.5.

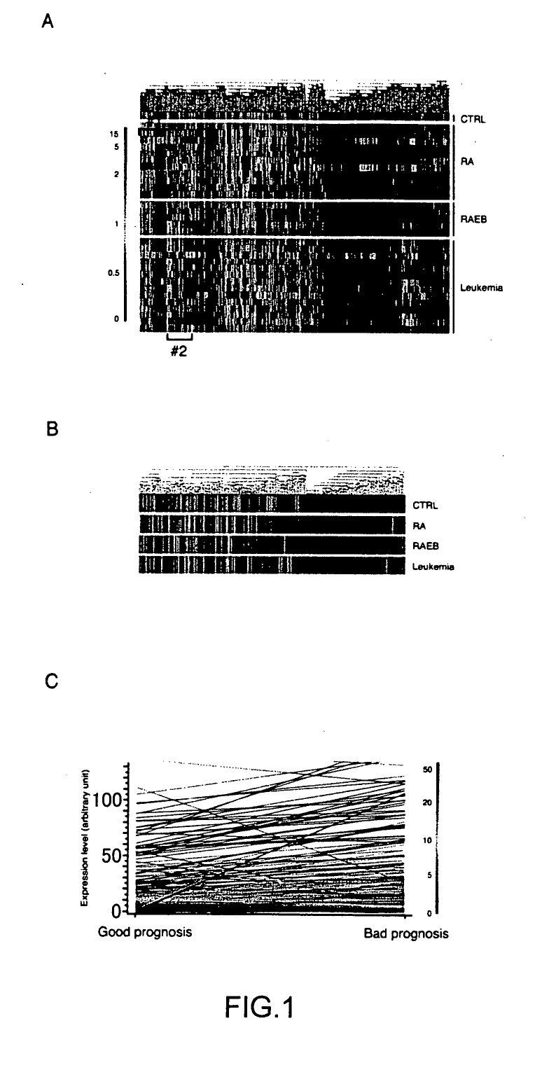

Expression intensity data of the genes were normalized relative to the median expression value of all genes in every hybridization. The resultant data was then used to generate a dendrogram, or “gene tree”, in which genes with similar expression profiles are clustered near each other. As shown in FIG. 1A, 50% of the genes on the array were transcriptionally silent throughout the clinical course of MDS. It is apparent, however, that there are some clusters, wherein the genes are expressed in a manner dependent to specific MDS stages. For instance, genes in the cluster #1 were highly expressed only in the RA patients #3 and #7, while those in the cluster #2 were more abundantly expressed in the samples of RAEB and MDS-associated leukemia compared to those from healthy control or RA. These genes should be potential molecular markers for each MDS-stage.

According to FIG. 1A, however, it was still difficult to precisely extract genes with stage-dependent expression profiles. For this purpose, the mean expression value among RA, RAEB or MDS-associated leukemia group was calculated for every gene on the array, and was used to generate another dendrogram, “average tree”. In FIG. 1B, readily identified were gene clusters with the stage-specific expression profiles. The inventor then tried to extract such genes through Welch ANOVA analysis. However, most genes thus identified as statistically “stage-dependent” (P<0.001) turned out not to be appropriate stage-specific markers. Such analysis had a preference to select genes whose expression level has a small deviation within each MDS stage. Unfortunately, the expression intensities for these genes did not largely change in a stage-dependent manner.

### EXAMPLE 2

### Genes Induced Along with Stage Progression

One of the main goals in this study was to clarify the molecular events that drive the stage progression from the indolent RA to the therapy-refractory RAEB/MDS-associate leukemia. The inventor thus focused to elucidate the change of transcriptomes between the good prognosis stages (healthy control and RA) and the bad prognosis stages (RAEB and MDS-associated leukemias). The mean expression intensity for every gene was calculated within the good or bad prognosis group, and used to draw FIG. 1C to demonstrate the change of expression level between the two groups for every gene.

The inventor first tried to isolate genes, expression of which was induced in the AC133⁺ cells of the bad prognosis group compared to those of the good prognosis one. Toward this goal, with the help of GeneSpring software (Silicon Genetics), the inventor searched for any genes whose expression profiles were statistically similar, with a minimum correlation of 0.99, to a hypothesized “bad prognosis-specific gene” (the blue line in FIG. 1C) that has a mean expression level of 0.0 arbitrary units (U) in the good prognosis stage but of 100.0 U in the bad prognosis one. From a total of 96 such genes, further selected were the genes whose expression values were kept below 20.0 U in all samples from the good prognosis group, but exceeded 50.0 U in at least one sample within the bad prognosis group.

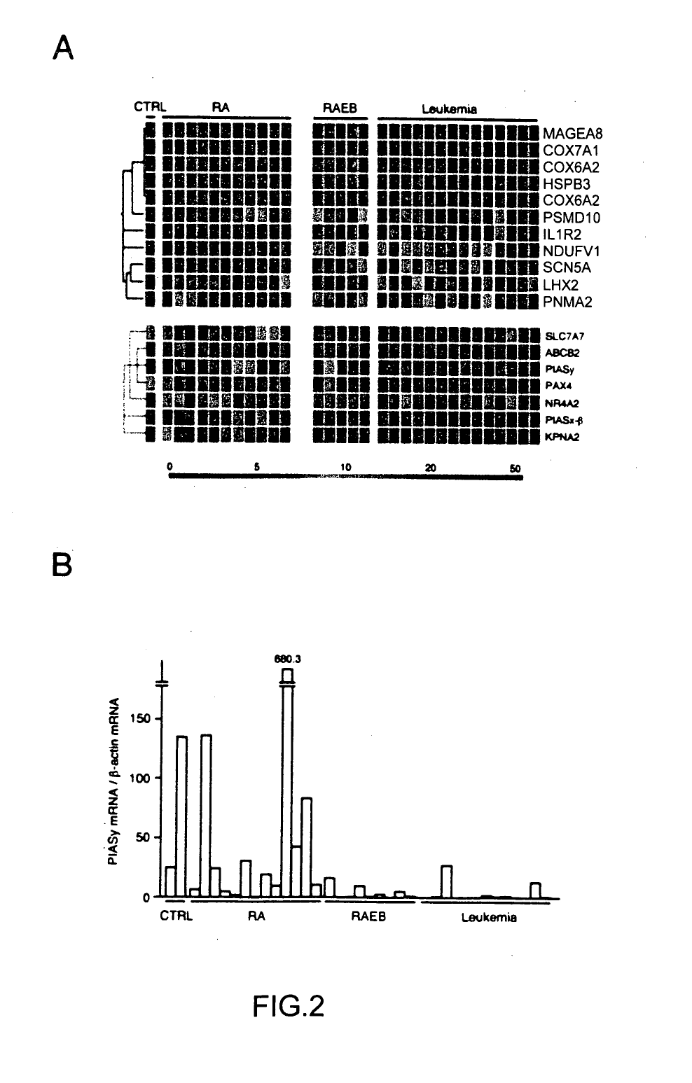

Finally, eleven such genes were identified (purple-colored gene tree in FIG. 2A), including those for NADH-ubiquinone oxidoreductase flavoprotein 1 (NDUFV1), LIM-Hox2 (LH2) and paraneoplastic antigen MA2 (PNMA2). Expression of the NDUFV1 gene was, for example, highly specific to the bad prognosis group. Its expression level was 4.60 U for the control sample and 1.50 U±0.92 (mean±SD) for the RA ones. However, its signal intensity raised up to 26.29 U±11.30 and 10.36 U±9.04, for the RAEB and MDS-associated leukemia samples, respectively. The difference of the NDUFV1 expression level between the good and bad prognosis groups was statistically significant (P=0.0061).

Expression of these genes only in the advanced stages of MDS may provide novel molecular markers useful for the differential diagnosis of MDS.

### EXAMPLE 3

### Genes Suppressed Upon Stage Progression

Given the essential roles of functional deficiency for various tumor-suppressors in the malignant transformation process, decrease of expression in certain genes may directly contribute to the stage progression in MDS as well. The inventor thus searched for any genes whose expression profiles were statistically similar to a hypothesized “good prognosis-specific gene” that has a mean expression level of 100.0 U in the good prognosis stage but of 0.0 U in the bad prognosis one. 182 such genes were identified, and further, genes whose expression values exceeded 70.0 U in at least one sample within the good prognosis group, and with those kept below 30.0 U in the bad prognosis group were selected, to finally identify 7 genes (blue-colored gene tree in FIG. 2A).

Among these control/RA-specific genes, of great interest would be those for PIAS family of signaling proteins, PIASy and PIASx-β (Shuai, K. (2000) Oncogene 19, 2638-2644). The present data demonstrates that the PIASy gene is activated in normal HSC or pluripotent stem cells in the indolent stage of MDS, but becomes suppressed upon the transition to the advanced stages. If the expression of PIASy is required in vivo to suppress de-regulated growth of HSC in normal or RA individuals, disappearance of its expression may allow the accelerated growth of blastic cells within BM, the hallmark of RAEB and MDS-associated leukemia. Therefore, expression level of PIASy may not only be useful as a molecular marker for stage diagnosis or prognosis estimation, but also be directly involved in the transformation mechanism toward advanced stages in MDS.

The present inventor thus paid attention to confirm the stage-dependent expression of PIASy gene by quantitative real-time PCR conducted as follows. Portions of unamplified cDNA were subjected to PCR with SYBR Green PCR Core Reagents (PE Applied Biosystems, Foster City, Calif.). The incorporation of the SYBR Green dye into the PCR products was monitored in real time with an ABI PRISM 7700 sequence detection system (PE Applied Biosystems), thereby allowing determination of the threshold cycle (C_{T}) at which exponential amplification of PCR products begins. The C_{T} values for cDNAs corresponding to the β-actin gene and PIASY were used to calculate the abundance of the PIASY transcript relative to that of β-actin mRNA. The polynucleotide primers for PCR were as follows: 5′-CCATCATGAAGTGTGACGTGG-3′ (SEQ ID NO: 1) and 5′-GTCCGCCTAGAAGCATTTGCG-3′ (SEQ ID NO: 2) for β-actin cDNA, 5′-AACTACGGCAAGAGCTACTCGGTG-3′ (SEQ ID NO: 3) and 5′-GTTCATCTGCAGGTAGAAGACGGC-3′ (SEQ ID NO: 4) for PIASycDNA.

The abundance of PIASy transcripts was determined relative to that of β-actin mRNA in the AC133+blasts from 37 individuals consisting of 2 healthy volunteers, 13 RA, 9 RAEB and 13 MDS-associated leukemia patients. Consistent with the results obtained by microarray analysis, the expression of the PIASy gene was high in the blasts from control or RA patients, but became markedly suppressed in those from the other individuals (P=0.043) (FIG. 2B). Thus the PIASY gene is a good candidate for the stage-dependent molecular markers in MDS.

### EXAMPLE 4

### Suppression of Cell Growth in 32D Cells Expressing PIASy

Given the possible proapoptotic activity (function as a tumor-suppressor) of PIASy, sustained expression of this gene may influence the growth or differentiation property of immature blood cells. To directly assess the effect of PIASy expression in the blood cell system, the present inventor utilized the pMX-tetOFF retrovirus vector that allows the dual regulation system with tetracycline and β-estradiol for the expression of an exogenous gene, and that constitutively expresses a gene to confer resistance against a selection marker drug, blasticidin-S (Iida, A., et al. (1996) J. Virol. 70, 60545-66059; Ohmine, K., et al. (2001) Oncogene 20, 8249-8257).

A mouse myeloid cell line, 32D (Greenberger, J. S., et al. (1983) Proc. Natl. Acad. Sci. USA 80, 2931-2935), grows without differentiation in response to IL-3, but undergoes terminal differentiation toward neutrophils by the stimulation with G-CSF. Due to this cytokine-dependent property, both growth and differentiation processes can be assayed in a single cell line, and therefore 32D cells were chosen for the recipient of the following infection experiment. First, 32D was maintained in RPMI1640 medium (Life Technologies, Rockville, Md.) supplemented with 10% fetal bovine serum (FBS) and 25 U/ml of interleukin (IL)-3. For the induction of granulocyte-differentiation, cells were cultured in RPMI1640/FBS containing 1 ng/ml of granulocyte colony-stimulating factor (G-CSF). The cDNA encoding human PIASy (Helix Institute, Chiba, Japan) with a COOH-terminal FLAG epitope tag (PIASy-F) was inserted into the pMX-tetOFF retrovirus vector plasmid (Ohmine, K., et al. (2001) Oncogene 20, 8249-8257), giving rise to pMX-tetOFF/PIASy-F. With the use of pMX-tetOFF/PIASy-F, all the polyclonal blasticidin-S-resistant cells express FLAG-tagged PIASy by the induction with β-estradiol and absence of tetracycline. On the other hand, expression of PIASy-F is kept suppressed by the presence of tetracycline or the absence of β-estradiol. Only with one round of infection, MX-tetOFF/PIASy-F virus should confer conditional expression of PIASy-F in target cells. The pMX-tetOFF or pMX-tetOFF/PIASy-F was then transiently introduced into a packaging cell line, BOSC23 (Pear, W. S., et al. (1993) Proc. Natl. Acad. Sci. USA 90, 8392-8396), to produce an ecotropic retrovirus, MX-tetOFF or MX-tetOFF/PIASy-F, respectively.

Then, 32D cells were infected with the supernatant of BOSC23 cells for 24 hr under the presence of Retronectin (Takara Shuzo, Shiga, Japan). The selection drug-resistant mass culture of 32D cells infected with MX-tetOFF or MX-tetOFF/PIASy-F was either cultured under non-induced [tetracycline (+), β-estradiol (−)] or induced [tetracycline (−), β-estradiol (+)] condition. The cells were harvested and cultured in RPMI1640/FBS/IL-3 supplemented with 5 μg/ml of blasticidin-S (Funakoshi, Tokyo, Japan) and 1 μg/ml of tetracycline (Boehringer Mannheim, Mannheim, Germany). To induce the expression of PIASy-F, the culture medium was changed to RPMI1640/FBS with 2 μM of 17β-estradiol (Sigma, St. Louis, Mo.) and appropriate cytokines. Protein analysis was carried out as described previously (Ohmine, K., et al. (2001) Oncogene 20, 8249-8257). Total cell lysates (10 μg/lane) were separated through 7.5% SDS-PAGE and subjected to the immunoblot analysis with the antibodies to FLAG (Eastman Kodak, New Haven, Conn.).

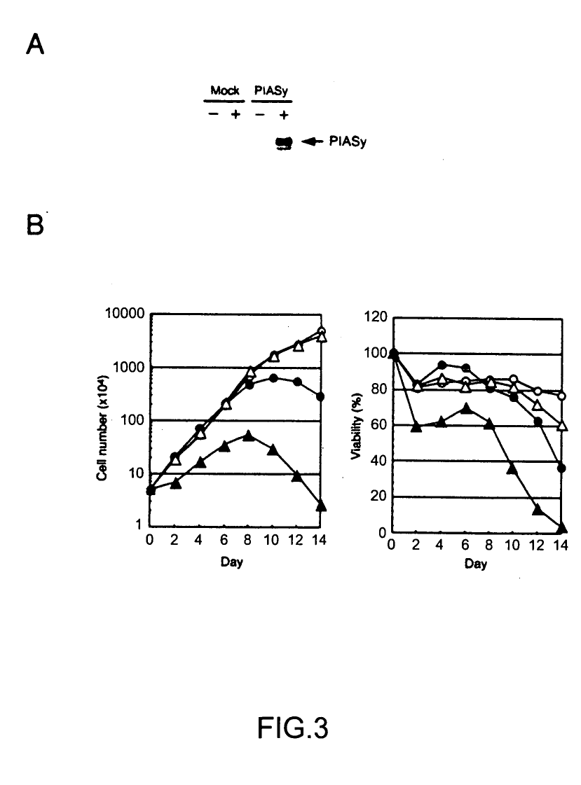

As shown in FIG. 3A, immunoblot analysis with antibodies to FLAG clearly demonstrated that PIASy was abundantly expressed only in the 32D cells infected with MX-tetOFF/PIASy-F under the induced condition. As expected, however, neither mock-infected 32D cells nor the MX-tetOFF/PIASy-F-infected 32D cells under non-induced condition expressed detectable amounts of PIASy-F.

To examine the influence of PIASy expression on cell growth, virus-infected 32D cells were cultured under the presence of IL-3. However, no difference in growth property, cell viability, cell-surface marker expression or cell shape in those between induced and non-induced conditions could be detected (not shown). By the stimulation with IL-3, 32D cells grew healthily irrespective of PIASy expression.

Under the presence of G-CSF and absence of IL-3, 32D cells start to differentiate to terminal granulocytes while growing slowly. Under the non-induced condition, both of mock-infected and MX-tetOFF/PIASy-F-infected cells grew in a similar manner (FIG. 3B, left panel). Surprisingly, however, induction of PIASy-F expression apparently suppressed the cell growth of 32D. Although β-estradiol itself has a weak suppressive effect on growth of the mock-infected cells (compare the open and closed circles), the effect on MX-tetOFF/PIASy-F-infected cells was far obvious. Concomitant with such growth suppression, viability of 32D cells clearly dropped when PIASy was induced (FIG. 3B, right panel).

Liu et al. have shown that proteins of PIAS family induce apoptosis through the activation of c-Jun NH₂-terminal kinase (JNK) 1 (Liu, B. & Shuai, K. (2001) J. Biol. Chem. 276, 36624-36631). However, in the case of 32D cells, no effect on the activity of JNK1 by the expression of PIASy could be detected. Furthermore, PIASy-mediated apoptosis was not prevented by the forced expression of a dominant-negative form of JNK1 (not shown). Therefore, it is likely that JNK1 is not a principle mediator for PIASy-driven apoptosis in our system.

### EXAMPLE 5

### PIASy Induces Apoptosis in 32D Cells

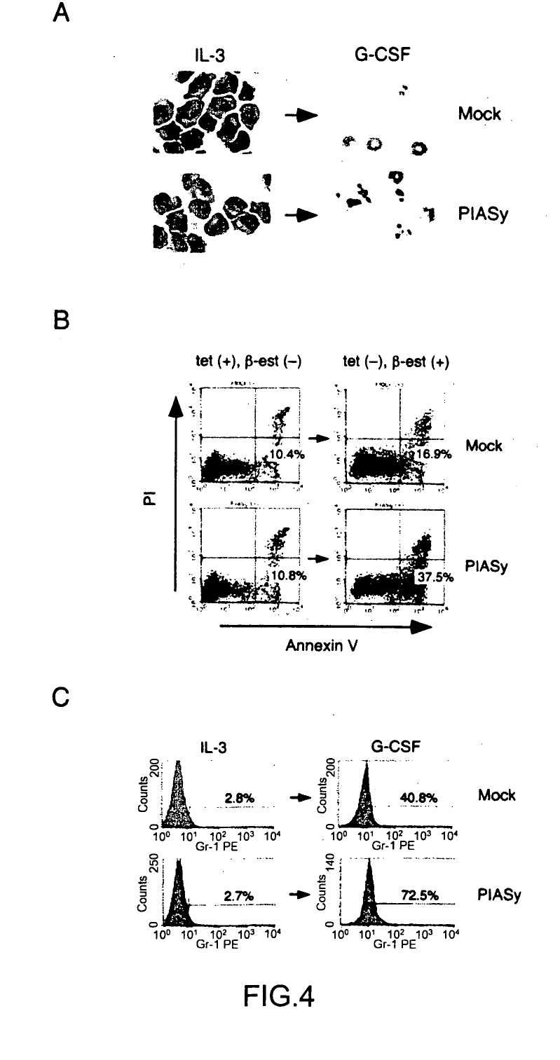

Retarded cell growth with the induction of cell death observed in FIG. 3 could be either the result of induction of apoptosis or that of acceleration in the differentiation process. To clarify the mechanism of growth suppression in 32D, morphology of the cells maintained for 8 days under the induced condition were analyzed by the staining with Wright-Giemsa solutions as follows. 32D cells infected with MX-tetOFF or MX-tetOFF/PIASy-F were cultured with G-CSF for 8 days under the presence of 17β-estradiol. Cytospin preparation of each cell fraction was stained with Wright-Giemsa solutions. Mock-infected cells under the presence of IL-3 were middle- to large-sized shape with a high nuclear to cytoplasm ratio (FIG. 4A). When cultured for 8 days with G-CSF, >50% of cells showed differentiated phenotypes; some cells with a lower nuclear to cytoplasm ratio and neutrophilic cytoplasm, and others with segmented nucleus.

Under the growing state with IL-3, the cells infected with MX-tetOFF/PIASy-F showed similar phenotype to that in the mock-infected cells. When cultured with G-CSF, however, there existed a marked increase in the number of cells with characteristic morphology of apoptosis; condensation and fragmentation of nuclei, and shrinkage of cells.

To directly estimate the extent of apoptosis in 32D cells under differentiation condition, the cells cultured with G-CSF for 8 days were stained with Annexin-V (BD Biosciences) and propidium iodide (PI), and subjected to flow cytometry analysis with a FACScan processor (BD Biosciences) to detect the translocation of phosphatidylserine (PS) from the inner part to the outer layer of cell membrane, a characteristic feature of apoptosis. Differentiation of 32D cells was evaluated by the FACScan processor with the antibodies to a granulocyte-specific marker, Gr-1 (BD Biosciences). As shown in FIG. 4B, incubation with G-CSF induced 10.4% or 16.9% of apoptotic cells (PS-positive and PI-negative) in the mock-infected 32D under non-induced or induced condition, respectively. Interestingly, PIASy expression increased the proportion of apoptotic cells from 10.8% to 37.5% in 32D cells incubated with G-CSF. The proportion of all dead cells (both of PS-positive/PI-negative and PS-positive/PI-positive) was 15.5%, 23.8%, 18.4% or 49.8% for the non-induced mock-infected cells, induced mock-infected cells, non-induced MX-tetOFF/PIASy-F-infected cells or induced MX-tetOFF/PIASy-F-infected cells, respectively. These data support the scenario in which PIASy suppresses growth of 32D cells under differentiation process through the induction of apoptosis.

To address whether PIASy accelerates the granulocyte differentiation as well, the present inventor measured the proportion of differentiated cells, under induced condition, by flow cytometry with antibodies against a granulocyte-specific cell surface protein, Gr-1 (BD Biosciences). When cultured under the presence of IL-3, proportion of Gr-1+cells was <3.0% both in mock- and MX-tetOFF/PIASy-F-infected cells (FIG. 4C). Incubation with G-CSF, instead, resulted in the expansion of Gr-1+population both in mock-infected (40.8%) and MX-tetOFF/PIASy-F-infected (72.5%) cells. The increase of Gr-1⁺ cells in the PIASy-expressing population may indicate that PIASy augments granulocyte differentiation. However, detailed inspection of FIG. 4C reveals that the fluorescence intensities at the maximum cell number were similar between the mock- and MX-tetOFF/PIASy-F-infected cells. Rather, it seems that decrease of Gr-1⁻ fraction in PIASy-positive cells did contribute to the overall increase of Gr-1⁺ proportion (compare the upper and lower panels in the right half). Examination with Wright-Giemsa staining of cell morphology in a time-course manner also revealed that appearance of terminal granulocytes with segmented nuclei could be detected after the same incubation period with G-CSF between the two groups (not shown) The only difference in the morphology between the two was the absence or presence of a large number of apoptotic cells. Therefore, the inventor supposed that the principle mechanism for PIASy suppression of cell growth was the induction of apoptosis rather than the induction of cell differentiation.

Given the potentially suppressive activity on STAT proteins, PIASy may control cell viability via STAT. In the 32D cells, the gel shift assay could not reveal any suppression of STAT1 activity by the PIASy expression (not shown). More importantly, the fact that G-CSF mediated cell differentiation was not inhibited by PIASy in 32D (FIG. 4C) implies that the intracellular signaling pathway through G-CSF receptor/STAT1 was intact.

Recently, several groups have revealed a catalytic activity of PIAS proteins for SUMO-conjugation. Both of PIASy and PIAS1 were shown to function as SUMO E3 ligase (Sachdev, S., et al. (2001) Genes Dev. 15, 3088-3103; Kahyo, T., et al. (2001) Mol. Cell 8, 713-718), and so was a yeast PIAS-related protein (Johnson, E. S. & Gupta, A. A. (2001) Cell 106, 735-744; Takahashi, Y., et al. (2001) J. Biol. Chem. 276: 48973-48977). SUMO is an ubiquitin-like molecule that is conjugated toward a variety of proteins such as RanGAP1, PML, IκBα and p53 (Hay, R. T. (2001) Trends Biochem. Sci. 26, 332-333). Although SUMO-conjugation seems to promote proteolysis of targets as the ubiquitin-system does, sumoylation may also have a direct regulatory role for the target proteins. Given the wide spectrum of sumoylated proteins, phenotypes or molecular events so far assigned to PIASy expression may be the results of PIASy-mediated sumoylation reactions. The final output or phenotype by PIASy-expression should thus be dependent on the character of PIASy substrates. It would be therefore indispensable to identify the binding partners of PIASy in 32D cells to decipher the molecular mechanism for PIASy-mediated apoptosis.

1. A method for identifying a myelodysplastic syndrome-specific gene, said method comprising the steps of:
(a) detecting gene expressions in hematopoietic stem cells prepared from a myelodysplastic syndrome patient at a bad prognosis stage and a normal individual or patient at a good prognosis stage;
(b) comparing the gene expression in the hematopoietic stem cells between the myelodysplastic syndrome patient at a bad prognosis stage and the normal individual or patient at a good prognosis stage; and
(c) identifying a gene specifically overexpressed or underexpressed in the myelodysplastic syndrome patient at a bad prognosis stage.

2. The method of claim 1, wherein the hematopoietic stem cells are prepared from bone marrow aspirate.

3. The method of claim 1, wherein the hematopoietic stem cells are prepared using hematopoietic stem cell-specific cell surface marker as an index.

4. A method of diagnosis for myelodysplastic syndrome, said method comprising the steps of:
(a) detecting, in tissue or cells prepared from a subject, expression of a myelodysplastic syndrome-specific gene identified by the method of claim 1; and
(b) comparing the expression detected in step (a) with that in a control tissue or control cells,
wherein the subject is judged to be at a risk of myelodysplastic syndrome (1) if the expression detected in step (a) is significantly higher than the expression of the gene in the control tissue or control cells, where the gene is specifically expressed in a myelodysplastic syndrome patient, or (2) if the expression detected in step (a) is significantly lower than that in the control tissue or control cells, where the gene is specifically expressed in individuals free from myelodysplastic syndrome.

5. The method of claim 1, wherein the myelodysplastic syndrome-specific gene is selected form the group of PIASy gene, LIM2 gene, NDUFV1 gene and PNMA2 gene.

6. The method of claim 4 or 5, wherein the cells prepared from the subject are hematopoietic stem cells.

7. The method of claim 6, wherein the hematopoietic stem cells are prepared from bone marrow aspirate.

8. The method of claim 6, wherein the hematopoietic stem cells are prepared using hematopoietic stem cell-specific cell surface marker as an index.

9. A drug for diagnosing myelodysplastic syndrome, said drug comprising, as an active ingredient, a molecule selected from the group consisting of:
(a) an antibody binding to a protein encoded by a myelodysplastic syndrome-specific gene identified by the method of claim 1; and
(b) a polynucleotide specifically hybridizing to a transcription product of a myelodysplastic syndrome-specific gene identified by the method of claim 1.

10. The drug of claim 9, wherein the myelodysplastic syndrome-specific gene is selected form the group of PIASy gene, LIM2 gene, NDUFV1 gene and PNMA2 gene.

11. A method of diagnosis for myelodysplastic syndrome, said method comprising the step of:
(a) detecting, in a biological sample, a genetic polymorphism or mutation that causes abnormal expression of a myelodysplastic syndrome-specific gene identified by the method of claim 1 or abnormal activity of a protein encoded by the gene;
wherein a subject from whom the biological sample was taken is judged to be at a risk of myelodysplastic syndrome if the genetic polymorphism or mutation has been detected.

12. The method of claim 11, wherein the myelodysplastic syndrome-specific gene is selected form the group of PIASy gene, LIM2 gene, NDUFV1 gene and PNMA2 gene.

13. A method for identifying compounds for treating or preventing myelodysplastic syndrome, said method comprising the steps of:
(a) administering or contacting a test compound to a test animal; and
(b) detecting, in the test animal or test cells, expression of a myelodysplastic syndrome-specific gene identified by the method of claim 1;
wherein the test compound is judged to be a drug candidate compound for treating or preventing myelodysplastic syndrome if the test compound decreases the expression detected in step (b), where the gene is specifically expressed in a myelodysplastic syndrome patient, or if the test compound increases the expression detected in step (b), where the gene is specifically expressed in individuals free from myelodysplastic syndrome.

14. A method for identifying compounds for treating or preventing myelodysplastic syndrome, said method comprising the steps of:
(a) administering or contacting a test compound to a test animal or test cells harboring a reporter gene operably linked to the expression control region of a myelodysplastic syndrome-specific gene identified by the method of claim 1; and
(b) detecting, in the test animal or test cells, expression of the reporter gene;
wherein the test compound is judged to be a drug candidate compound for treating or preventing myelodysplastic syndrome (1) if the test compound decreases the expression detected in step (b), where the myelodysplastic syndrome-specific gene is specifically expressed in a myelodysplastic syndrome patient, or (2) if the test compound increases the expression detected in step (b), where the gene is specifically expressed in individuals free from myelodysplastic syndrome.

15. A method for identifying compounds for treating or preventing myelodysplastic syndrome, said method comprising the steps of:
(a) contacting a test compound with a protein encoded by a myelodysplastic syndrome-specific gene identified by the method of claim 1; and
(b) detecting activity of the protein;
wherein the test compound is judged to be a drug candidate compound for treating or preventing myelodysplastic syndrome (1) if the test compound decreases the activity detected in step (b), where the gene is specifically expressed in a myelodysplastic syndrome patient, or (2) if the test compound increases the activity detected in step (b), where the gene is specifically expressed in individuals free from myelodysplastic syndrome.

16. The method of any one of claims 13 to 15, wherein the myelodysplastic syndrome-specific gene is selected form the group of PIASy gene, LIM2 gene, NDUFV1 gene and PNMA2 gene.

17. A drug for treating or preventing myelodysplastic syndrome, said drug comprising, as an active ingredient, a compound identified by the method of any one of claims 13 to 16.


SEQUENCE LISTING (4 sequences)

SEQ ID NO: 1 - DNA, 21 bp, Artificial Sequence
  synthetic oligonucleotide
ccatcatgaagtgtgacgtgg

SEQ ID NO: 2 - DNA, 21 bp, Artificial Sequence
  synthetic oligonucleotide
ctccgcctagaagcatttgcg

SEQ ID NO: 3 - DNA, 24 bp, Artificial Sequence
  synthetic oligonucleotide
aactacggcaagagctactcggtg

SEQ ID NO: 4 - DNA, 24 bp, Artificial Sequence
  synthetic oligonucleotide
gttcatctgcaggtagaagacggc